(19) United States
(12) Patent Application Publication
(10) Pub. No.: US 2016/0122774 A1
(43) Pub. Date: May 5, 2016

(54) A METHOD FOR PRODUCING PRECISE DNA CLEAVAGE USING CAS9 NICKASE ACTIVITY

(71) Applicant: CELLECTIS, Paris (FR)

(72) Inventors: Philippe DUCHATEAU, Draveil (FR); Claudia BERTONATI, Paris (FR)

(21) Appl. No.: 14/892,743

(22) PCT Filed: May 28, 2014
(86) PCT No.: PCT/EP2014/061178

§ 371 Date: Nov. 20, 2015

(30) Foreign Application Priority Data
May 29, 2013 (DK) PA201370295

(51) Int. Cl. C12N 15/82; C12N 15/90

(52) U.S. Cl. CPC C12N 15/8213; C12N 15/907

(57) ABSTRACT

The present invention is in the field of a method for genome engineering based on the type II CRISPR system, particularly a method for improving specificity and reducing potential off-site. The method is based on the use of nickase architectures of Cas9 and single or multiple crRNA(s) harboring two different targets lowering the risk of producing off-site cleavage. The present invention also relates to polypeptides, polynucleotides, vectors, compositions, therapeutic applications related to the method described here.

### FIELD OF THE INVENTION

The present invention relates to a method of genome engineering based on the type II CRISPR system. In particular, the invention relates to a method for precisely inducing a nucleic acid cleavage in a genetic sequence of interest and preventing off-site cleavage. The method is based on the use of nickase architectures of Cas9 and single or multiple crRNA(s) harboring two different targets lowering the risk of producing off-site cleavage. The present invention also relates to polypeptides, polynucleotides, vectors, compositions, therapeutic applications related to the method described here.

### BACKGROUND OF THE INVENTION

Site-specific nucleases are powerful reagents for specifically and efficiently targeting and modifying a DNA sequence within a complex genome. There are numerous applications of genome engineering by site-specific nucleases extending from basic research to bioindustrial applications and human therapeutics. Re-engineering a DNA-binding protein for this purpose has been mainly limited to the design and production of proteins such as the naturally occurring LADLIDADG homing endonucleases (LHE), artificial zinc finger proteins (ZFP), and Transcription Activator-Like Effectors nucleases (TALE-nucleases).

Recently, a new genome engineering tool has been developed based on the RNA-guided Cas9 nuclease (Gasiunas, Barrangou et al. 2012; Jinek, Chylinski et al. 2012) from the type II prokaryotic CRISPR (Clustered Regularly Interspaced Short palindromic Repeats) adaptive immune system. The CRISPR Associated (Cas) system was first discovered in bacteria and functions as a defense against foreign DNA, either viral or plasmid. So far three distinct bacterial CRISPR systems have been identified, termed type I, II and III. The Type II system is the basis for the current genome engineering technology available and is often simply referred to as CRISPR. The type II CRISPR/Cas loci are composed of an operon of genes encoding generally the proteins Cas9, Cas1, Cast and Csn2a, Csn2bor Cas4 (Chylinski, Le Rhun et al. 2013), a CRISPR array consisting of a leader sequence followed by identical repeats interspersed with unique genome-targeting spacers and a sequence encoding the trans-activating tracrRNA.

CRISPR-mediated adaptative immunity proceeds in three distinct stages: acquisition of foreign DNA, CRISPR RNA (crRNA) biogenesis and target interference. (see review (Sorek, Lawrence et al. 2013)). First, the CRISPR/Cas machinery appears to target specific sequence for integration into the CRISPR locus. Sequences in foreign DNA selected for integration are called spacers and these sequences are often flanked by a short sequence motif, referred as the proto-spacer adjacent motif (PAM). crRNA biogenesis in type II systems is unique in that it requires a trans-activating crRNA (tracRNA). CRISPR locus is initially transcribed as long precursor crRNA (pre-crRNA) from a promoter sequence in the leader. Cas9 acts as a molecular anchor facilitating the base pairing of tracRNA with pre-cRNA for subsequent recognition and cleavage of pre-cRNA repeats by the host RNase III (Deltcheva, Chylinski et al. 2011). Following the processing events, tracrRNA remains paired to the crRNA and bound to the Cas9 protein. In this ternary complex, the dual tracrRNA:crRNA structure acts as guide RNA that directs the endonuclease Cas9 to the cognate target DNA (Jinek, Chylinski et al. 2012). Target recognition by the Cas9-tracrRNA:crRNA complex is initiated by scanning the invading DNA molecule for homology between the protospacer sequence in the target DNA and the spacer-derived sequence in the crRNA. In addition to the DNA protospacer-crRNA spacer complementarity, DNA targeting requires the presence of a short motif adjacent to the protospacer (protospacer adjacent motif—PAM). Following pairing between the dual-RNA and the protospacer sequence, Cas9 subsequently introduces a blunt double strand break 3 bases upstream of the PAM motif (Garneau, Dupuis et al. 2010).

The large Cas9 protein (>1200 amino acids) contains two predicted nuclease domains, namely HNH (McrA-like) nuclease domain that is located in the middle of the protein and a splitted RuvC-like nuclease domain (RNase H fold) (Haft, Selengut et al. 2005; Makarova, Grishin et al. 2006). The HNH nuclease domain and the Ruv-C domain have been found to be essential for double strand cleavage activity. Mutations introduced in these domains have respectively led to Cas9 proteins displaying nickase-activity instead of double-strand cleavage activity. Different inactivating mutation(s) of the catalytic residues in the RuvC-like domains produces a nickase able to cut one strandin position +3 bp (versus the 3′ end) respect with the PAM location. The mutation of the catalytic residue of the HNH domain generates a nickase able to cut the other strandin position +3 bp (versus the 5′ end) (Jinek, Chylinski et al. 2012) (FIG. 1).

Prokaryote type II CRISPR system is capable of recognizing any potential target sequence of 12 to 20 nucleotides followed by a specific PAM motif on its 3′ end. However, the specificity for target recognition relies on only 12 nucleic acids (Jiang, Bikard et al. 2013; Qi, Larson et al. 2013), which is enough for ensuring unique cleavage site prokaryotic genomes on a statistical basis, but which is critical for larger genomes, like in eukaryotic cells, where 12 nucleic acids sequences may be found several times. There is therefore a need to develop strategies for improving specificity and reducing potential off-site using type II CRISPR system.

### SUMMARY OF THE INVENTION

Here the inventors have investigated different modifications into type II CRISPR system for improving specificity and reducing potential off-site. Unexpectedly, they found that using mutated version of Cas9 having nickase activity, instead of cleavase activity, can be used to produce cleavage at a given DNA target and increase the specificity in the same time. The method is based on the simultaneous use of nickase architecture of Cas9 (RuvC domain and/or HNH domain) and sgRNA(s) harboring two different complementary sequence to specific targets lowering the risk of producing off-site cleavage. By using at least one guide RNA harboring two different complementary sequence to specific targets or a combination of at least two guide RNA, the requirement for specificity passes from 12 to 24 nucleotides and, in turn, the probability to find two alternative binding sites of Cas9 (different from the ones coded in the two sgRNA) at an efficient distance from each other to produce an off-site cleavage gets really low. The invention extends to the crRNA, tracrRNA and Cas mutants designed to perform this method and to the cells transfected with the resulting modified type II CRISPR system.

### BRIEF DESCRIPTION OF THE FIGURES

FIG. 1: Schematic of the type II CRIPSR/Cas system mediated DNA double-strand break. In the type II CRISPR/Cas system, Cas 9 is guided by a two-RNA structure, named guide RNA (gRNA) formed by crRNA and tracRNA to cleave double-stranded nucleic acid target (dsDNA). Cas9 RuvC domain induces a nick event (arrow) in one strand in position +3 bp (versus the 3′ end) respect with the PAM location and the Cas9 HNH domain induces a nick event (arrow) in the other strand in position +3 bp (versus the 5′ end). For better understanding, the figure illustrates only one aspect of the CRISPR/Cas system mediated double-strand break.

FIG. 2: Schematic of the new type II CRISPR/Cas system using a Cas9 nickase. A-B Two nucleic acid targets each comprising in one strand a PAM motif in the 3′-ends are selected within a genetic sequence of interest. The two nucleic acid targets are spaced by a distance “d”. Cas9 harboring a non-functional RuvC or HNH domain (RuvC(−) (A-C) or (HNH(−) (B-D) respectively) is guided by two engineered gRNA each comprising a sequence complementary to at least 12 nucleotides adjacent to the complementary PAM motif of the first and second nucleic acid targets. A-B. Each PAM motifs of the two targets are present in different strands. The Cas9 nickase induces a nick (arrow) in the different strands resulting in a double-strand break within the genetic sequence of interest. C-D. Each PAM motifs of the two targets are present in the same strand. The Cas9 nickase induces a nick in the same strand of the genetic sequence of interest, resulting in the deletion of a single-strand nucleic acid sequence between the two nick events. The figure illustrates only some aspects of the CRISPR/Cas system using Cas9 nickase.

FIG. 3: Schematic of the new type II CRISPR/Cas system using two different Cas9 nickases. A-B Two nucleic acid targets comprising two different PAM motifs (PAM1 and PAM2) in the 3′ end are selected within a genetic sequence of interest. The two nucleic acid targets are spaced by a distance “d”. A first Cas9 harboring a non-functional RuvC is guided by an engineered gRNA which comprises sequence complementary to at least 12 nucleotides adjacent to the first complementary PAM motif. A second Cas9 harboring a non-functional HNH domain is guided by a second engineered gRNA which comprises a sequence complementary to at least 12 nucleotides adjacent to the second complementary PAM motif. A. Each PAM motifs of the two targets are present in the different strands. The two Cas9 nickases induce two nicks (arrows) in the same strand of the genetic sequence of interest, resulting in the deletion of a single-strand nucleic acid sequence between the two nick events. B. Each PAM motifs of the two targets are present in the same strand. The Cas9 nickases induce two nick events (arrows) in the different strands resulting in a double-strand break within the genetic sequence of interest. The figure illustrates only some aspects of the CRISPR/Cas system using Cas9 nickase.

### DISCLOSURE OF THE INVENTION

Unless specifically defined herein, all technical and scientific terms used have the same meaning as commonly understood by a skilled artisan in the fields of gene therapy, biochemistry, genetics, molecular biology and immunology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N. Y., 1986).

### Method for Precisely Inducing a Nucleic Acid Cleavage in a Genetic Sequence

The present invention thus relates to a new method based on the CRISPR/Cas system to precisely induce a cleavage in a double-stranded nucleic acid target. This method derives from the genome engineering CRISPR adaptive immune system tool that has been developed based on the RNA-guided Cas9 nuclease (Gasiunas, Barrangou et al. 2012; Jinek, Chylinski et al. 2012).

In a more particular embodiment, the present invention relates to a method for precisely inducing nucleic acid cleavage in a genetic sequence in a cell comprising one of several of the following steps:
(a) Selecting a first and second double-stranded nucleic acid targets in said genetic sequence, each nucleic acid targets comprising, on one strand, a PAM motif at one 3′ extremities;
(b) engineering two crRNAs comprising each:
   a sequence complementary to one part of the opposite strand of the nucleic acid target that does not comprise the PAM motif, and
   a 3′ extension sequence;
(c) providing at least one tracrRNA comprising a sequence complementary to one part of the 3′ extension sequences of said crRNAs under b);
(d) providing at least one cas9 nickase specifically recognizing said PAM motif(s);
(e) introducing into the cell said crRNAs, said tracrRNA(s) and said Cas9 nickase;
   such that each Cas9-tracrRNA:crRNA complex induces a nick event in double-stranded nucleic acid targets in order to cleave the genetic sequence between said nucleic acid targets.

Said cleavage can result from at least one nick event in one nucleic acid strand, preferably two nicks events in the same nucleic acid strand or more preferably two nick events on the opposite nucleic acid strands.

Cas9, also named Csn1 (COG3513—SEQ ID NO: 1) is a large protein that participates in both crRNA biogenesis and in the destruction of invading DNA. Cas9 has been described in different bacterial species such as S. thermophilus (Sapranauskas, Gasiunas et al. 2011), listeria innocua (Gasiunas, Barrangou et al. 2012; Jinek, Chylinski et al. 2012) and S. Pyogenes (Deltcheva, Chylinski et al. 2011). The large Cas9 protein (>1200 amino acids) contains two predicted nuclease domains, namely HNH (McrA-like) nuclease domain that is located in the middle of the protein and a splitted RuvC-like nuclease domain (RNase H fold) (Haft, Selengut et al. 2005; Makarova, Grishin et al. 2006).

HNH motif is characteristic of many nucleases that act on double-stranded DNA including colicins, restriction enzymes and homing endonucleases. The domain HNH (SMART ID: SM00507, SCOP nomenclature:HNH family) is associated with a range of DNA binding proteins, performing a variety of binding and cutting functions (Gorbalenya 1994; Shub, Goodrich-Blair et al. 1994). Several of the proteins are hypothetical or putative proteins of no well-defined function. The ones with known function are involved in a range of cellular processes including bacterial toxicity, homing functions in groups I and II introns and inteins, recombination, developmentally controlled DNA rearrangement, phage packaging, and restriction endonuclease activity (Dalgaard, Klar et al. 1997). These proteins are found in viruses, archaebacteria, eubacteria, and eukaryotes. Interestingly, as with the LAGLI-DADG and the GIY-YIG motifs, the HNH motif is often associated with endonuclease domains of self-propagating elements like inteins, Group I, and Group II introns (Gorbalenya 1994; Dalgaard, Klar et al. 1997). The HNH domain can be characterized by the presence of a conserved Asp/His residue flanked by conserved His (amino-terminal) and His/Asp/Glu (carboxy-terminal) residues at some distance. A substantial number of these proteins can also have a CX2C motif on either side of the central Asp/His residue. Structurally, the HNH motif appears as a central hairpin of twisted β-strands, which are flanked on each side by an a helix (Kleanthous, Kuhlmann et al. 1999). The other CRISPR catalytic domain RuvC like RNaseH (also named RuvC) is found in proteins that show wide spectra of nucleolytic functions, acting both on RNA and DNA (RNaseH, RuvC, DNA transposases and retroviral integrases and PIWI domain of Argonaut proteins).

Recently, it has been demonstrated that HNH domain is responsible for nicking of one strand of the target double-stranded DNA and the RuvC-like RNaseH fold domain is involved in nicking of the other strand (comprising the PAM motif) of the double-stranded nucleic acid target (Jinek, Chylinski et al. 2012). However, in wild-type Cas9, these two domains result in blunt cleavage of the invasive DNA within the same target sequence (proto-spacer) in the immediate vicinity of the PAM (Jinek, Chylinski et al. 2012). In the present invention, Cas 9 is a nickase and induces a nick event within different target sequences. As non-limiting example, Cas9 can comprise mutation(s) in the catalytic residues of either the HNH or RuvC-like domains, to induce a nick event within different target sequences. As non-limiting example, the catalytic residues of the compact Cas9 protein are those corresponding to amino acids D10, D31, H840, H868, N882 and N891 of SEQ ID NO: 1 or aligned positions using CLUSTALW method on homologues of Cas Family members. Any of these residues can be replaced by any other amino acids, preferably by alanine residue. Mutation in the catalytic residues means either substitution by another amino acids, or deletion or addition of amino acids that induce the inactivation of at least one of the catalytic domain of cas9. (cf (Sapranauskas, Gasiunas et al. 2011; Jinek, Chylinski et al. 2012). In a particular embodiment, Cas9 may comprise one or several of the above mutations. In another particular embodiment, Cas9 may comprise only one of the two RuvC and HNH catalytic domains. In the present invention, Cas9 of different species, Cas9 homologues, Cas9 engineered and functional variant thereof can be used. The invention envisions the use of such Cas9 variants to perform nucleic acid cleavage in a genetic sequence of interest. Said Cas9 variants have an amino acid sequence sharing at least 70%, preferably at least 80%, more preferably at least 90%, and even more preferably 95% identity with Cas9 of different species, Cas9 homologues, Cas9 engineered and functional variant thereof. Preferably, said Cas9 variants have an amino acid sequence sharing at least 70%, preferably at least 80%, more preferably at least 90%, and even more preferably 95% identity with SEQ ID NO: 1.

The nucleic acid cleavages caused by site-specific nucleases are commonly repaired through the distinct mechanisms of homologous recombination or non-homologous end joining (NHEJ). Although homologous recombination typically uses the sister chromatid of the damaged DNA as an exogenous nucleic acid sequence from which to perform perfect repair of the genetic lesion, NHEJ is an imperfect repair process that often results in changes to the DNA sequence at the site of the cleavage. Mechanisms involve rejoining of what remains of the two DNA ends through direct re-ligation (Critchlow and Jackson 1998) or via the so-called microhomology-mediated end joining (Ma, Kim et al. 2003). Also, repair via non-homologous end joining (NHEJ) often results in small insertions or deletions and can be used for the creation of specific gene knockouts. Thus, one aspect of the present invention is to induce knock-outs or to introduce exogenous genetic sequences by homologous recombination into specific genetic loci.

By genetic sequence of interest is meant any endogenous nucleic acid sequence, such as, for example a gene or a non-coding sequence within or adjacent to a gene, in which it is desirable modify by targeted cleavage and/or targeted homologous recombination. The sequence of interest can be present in a chromosome, an episome, an organellar genome such as mitochondrial or chloroplast genome or genetic material that can exist independently to the main body of genetic material such as an infecting viral genome, plasmids, episomes, transposons for example. A sequence of interest can be within the coding sequence of a gene, within transcribed non-coding sequence such as, for example, leader sequences, trailer sequence or introns, or within non-transcribed sequence, either upstream or downstream of the coding sequence.

The first and the second double-stranded nucleic acid targets are comprised within the genetic sequence of interest into which it is desired to introduce a cleavage and thus genetic modification. Said modification may be a deletion of the genetic material, insertion of nucleotides in the genetic material or a combination of both deletion and insertion of nucleotides. By “target nucleic acid sequence”, “double-stranded nucleic acid target” or “DNA target” is intended a polynucleotide that can be processed by the Cas9-tracrRNA:crRNA complex according to the present invention. The double-stranded nucleic acid target sequence is defined by the 5′ to 3′ sequence of one strand of said target. These terms refer to a specific DNA location within the genetic sequence of interest. The two targets can be spaced away each other from 1 to 500 nucleotides, preferably between 3 to 300 nucleotides, more preferably between 3 to 50 nucleotides, again more preferably between 1 to 20 nucleotides.

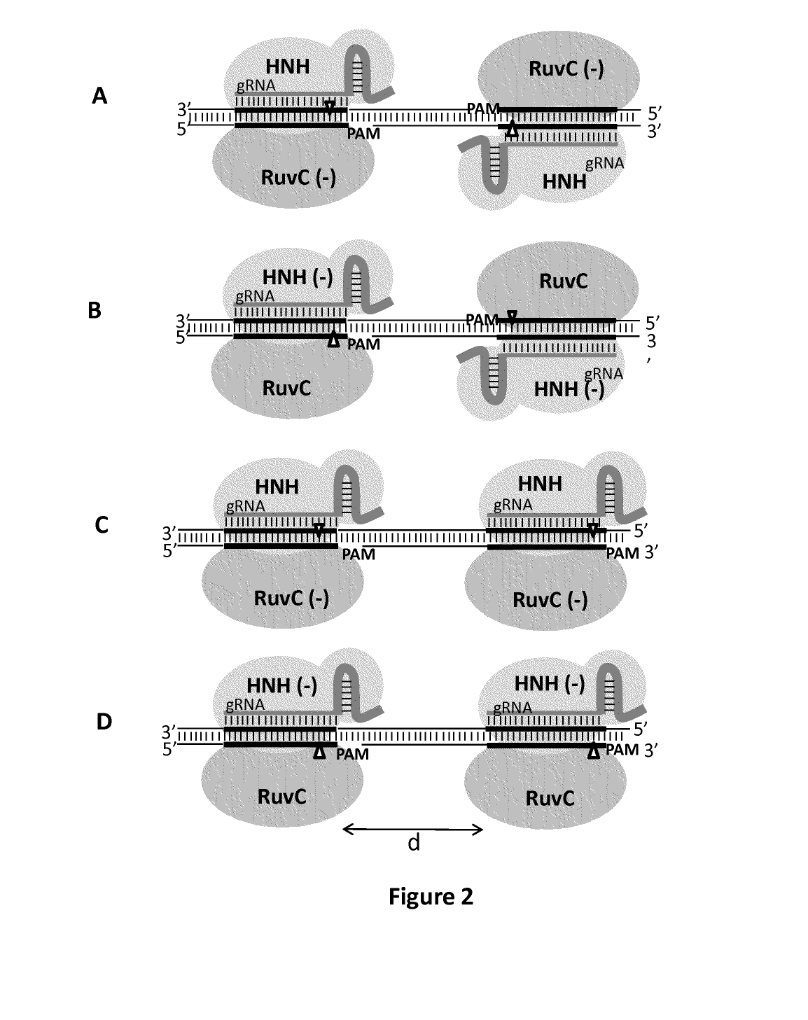

Any potential selected double-stranded DNA target in the present invention may have a specific sequence on its 3′ end, named the protospacer adjacent motif or protospacer associated motif (PAM). The PAM is present in the strand of the nucleic acid target sequence which is not complementary to the crRNA. Preferably, the proto-spacer adjacent motif (PAM) may correspond to 2 to 5 nucleotides starting immediately or in the vicinity of the proto-spacer at the 3′-end. The sequence and the location of the PAM motif recognized by specific Cas9 vary among the different systems. PAM motif can be for examples NNAGAA, NAG, NGG, NGGNG, AWG, CC, CCN, TCN, TTC as non limiting examples (Shah, Erdmann et al. 2013). Different Type II systems have differing PAM requirements. For example, the S. pyogenes system requires an NGG sequence, where N can be any nucleotides. S. thermophilus Type II systems require NGGNG (Horvath and Barrangou 2010) and NNAGAAW (Deveau, Barrangou et al. 2008), while different S. mutant systems tolerate NGG or NAAR (van der Ploeg 2009). PAM is not restricted to the region adjacent to the proto-spacer but can also be part of the proto-spacer (Mojica, Diez-Villasenor et al. 2009). In a particular embodiment, the Cas9 protein can be engineered to recognize a non-natural PAM motif. In this case, the selected target sequence may comprise a smaller or a larger PAM motif with any combinations of amino acids. As non-limiting example, the two PAM motifs of the two nucleic acid targets can be present on the same nucleic acid strand and thus the Cas9 nickase harboring a non-functional RuvC or HNH nuclease domain induces two nick events on the same strand (FIGS. 2C and D). In this case, the resulting single-strand nucleic acid located between the first and the second nick can be deleted. This deletion may be repaired by NHEJ or homologous recombination mechanisms. In another aspect of the invention, the two PAM motifs of the two nucleic acid targets can be present on opposed nucleic acid strands and thus the Cas9 nickase harboring a non-functional RuvC or HNH nuclease domain induces two nick events on each strand of the genetic sequence of interest (FIGS. 2A and B) resulting in a double strand break within the genetic sequence of interest.

In a particular embodiment, the method of the present invention used two Cas9 nickases, each one capable of recognizing different PAM motifs within the two nucleic acid targets. As non-limiting example, the first Cas9 is capable of recognizing the NGG PAM motif and the second Cas9 is capable of recognizing the NNAGAAW PAM motif.

In particular, the present invention relates to a method comprising one or several of the following steps:
(a) selecting a first and second double-stranded nucleic acid target sequences each comprising in one strand a PAM motif at their 3′ extremities, wherein said PAM motifs are different;
(b) engineering two crRNAs comprising each a sequence complementary to a part of the other strand of the first and second double-stranded nucleic acid targets and having a 3′ extension sequence;
(c) providing at least one tracrRNA comprising a sequence complementary to a part of the 3′ extension sequences of said crRNAs;
(d) providing a first cas9 nuclease specifically recognizing the PAM motif of the first target and harboring a non-functional RuvC-like or HNH nuclease domain;
(e) providing a second Cas9 specifically recognizing the PAM motif of the second target and harboring a non-functional RuvC-like or HNH nuclease domain;
(f) introducing into the cell said crRNAs, said tracrRNA(s), said Cas9 nucleases such that each Cas9-tracrRNA:crRNA complex induces a nick event in the double-stranded nucleic acid target.

As non-limiting examples, S. pyogenes Cas9 lacking functional RuvC or HNH catalytic domain and S. thermophilus Cas9 lacking functional RuvC or HNH catalytic domain can be introduced into the cell to specifically recognize NGG PAM motif in the first target nucleic acid sequence and NNAGAAW PAM motif in the second target nucleic acid sequence respectively. In particular embodiment, the two distinct PAM motifs of the two nucleic acid targets can be present on the same nucleic acid strand and thus the Cas9 nickases harboring a non-functional RuvC or HNH nuclease domain induces two nick events on the same strand. In this case, the resulting single-strand nucleic acid located between the first and the second nick can be deleted. In another embodiment, the two distinct PAM motifs of the two nucleic acid targets can be present on opposed nucleic acid strands and thus the Cas9 nickases harboring a non-functional RuvC-like or HNH nuclease domain induces two nick events on each strand of the genetic sequence of interest resulting in a double-strand break within the genetic sequence of interest.

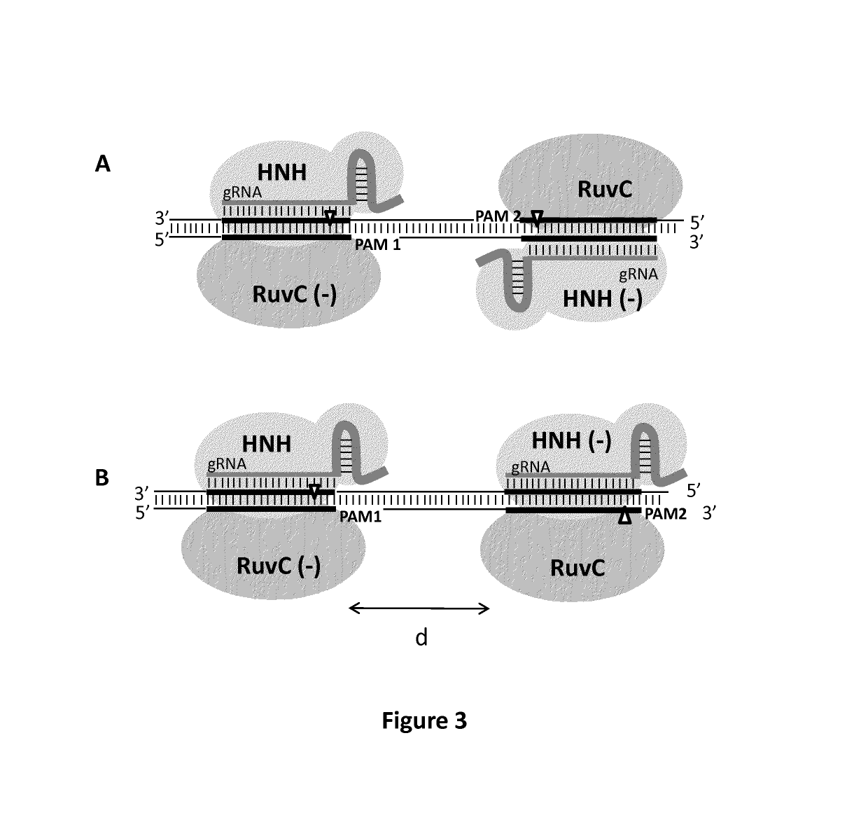

In another particular embodiment, the first Cas9 nickase harbors a non-functional RuvC-like nuclease domain and the second Cas9 nickase harbors a non-functional HNH nuclease domain. The different PAM motifs of the two nucleic acid targets can be on the same strand, thus the two Cas9 nickases induce a nick event on each strand (FIG. 3A), resulting in a double-strand break within the genetic sequence of interest. The two PAM motifs can also be on opposed strands and thus the two Cas9 nickases induce a nick event on the same strand of the genetic sequence of interest (FIG. 3B). In this case, the resulting single-strand nucleic acid located between the first and the second nick can be deleted. This deletion may be repaired by NHEJ or homologous recombination mechanisms.

The method of the present invention comprises engineering two crRNAs with distinct complementary regions to each nucleic acid target. In natural type II CRISPR system, the CRISPR targeting RNA (crRNA) targeting sequences are transcribed from DNA sequences known as protospacers. Protospacers are clustered in the bacterial genome in a group called a CRISPR array. The protospacers are short sequences of known foreign DNA separated by a short palindromic repeat and kept like a record against future encounters. To create the crRNA, the CRISPR array is transcribed and the RNA is processed to separate the individual recognition sequences between the repeats. The Spacer-containing CRISPR locus is transcribed in a long pre-crRNA. The processing of the CRISPR array transcript (pre-crRNA) into individual crRNAs is dependent on the presence of a trans-activating crRNA (tracrRNA) that has sequence complementary to the palindromic repeat. The tracrRNA hybridizes to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9 and form the Cas9-tracrRNA:crRNA complex. Engineered crRNA with tracrRNA is capable of targeting a selected nucleic acid sequence, obviating the need of RNase III and the crRNA processing in general (Jinek, Chylinski et al. 2012).

In the present invention, two crRNA are engineered to comprise distinct sequences complementary to a part of one strand of the two nucleic acid targets such that it is capable of targeting, preferably inducing a nick event in each nucleic acid targets. In particular embodiment, the two nucleic acid targets are spaced away each other from 1 to 300 bp, preferably from 3 to 250 bp, preferably from 3 to 200 bp, more preferably from 3 to 150 bp, 3 to 100 bp, 3 to 50 bp, 3 to 25 bp, 3 to 10 bp.

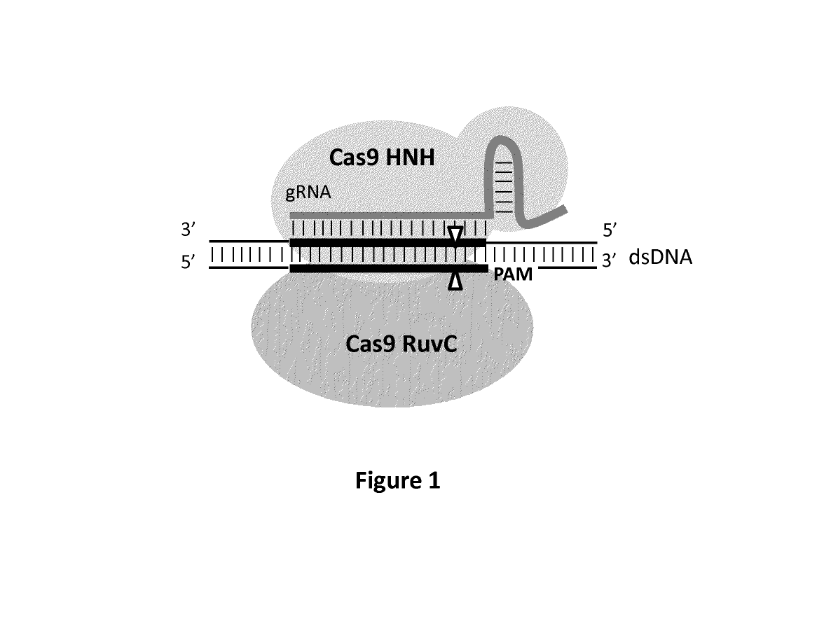

crRNA sequence is complementary to a strand of nucleic acid target, this strand does not comprise the PAM motif at the 3′-end (FIG. 1). In a particular embodiment, each crRNA comprises a sequence of 5 to 50 nucleotides, preferably 8 to 20 nucleotides, more preferably 12 to 20 nucleotides which is complementary to the target nucleic acid sequence. In a more particular embodiment, the crRNA is a sequence of at least 30 nucleotides which comprises at least 10 nucleotides, preferably 12 nucleotides complementary to the target nucleic acid sequence. In particular, each crRNA may comprise a complementary sequence followed by 4-10 nucleotides on the 5′ end to improve the efficiency of targeting (Cong, Ran et al. 2013; Mali, Yang et al. 2013; Qi, Larson et al. 2013). In preferred embodiment, the complementary sequence of the crRNA is followed in 3′-end by a nucleic acid sequences named repeat sequence or 3′ extension sequence.

The crRNA according to the present invention can also be modified to increase its stability of the secondary structure and/or its binding affinity for Cas9. In a particular embodiment, the crRNA can comprise a 2′,3′-cyclic phosphate. The 2′,3′-cyclic phosphate terminus seems to be involved in many cellular processes i.e. tRNA splicing, endonucleolytic cleavage by several ribonucleases, in self-cleavage by RNA ribozyme and in response to various cellular stress including accumulation of unfolded protein in the endoplasmatic reticulum and oxidative stress (Schutz, Hesselberth et al. 2010). The inventors have speculated that the 2′,3′-cyclic phosphate enhances the crRNA stability or its affinity/specificity for Cas9. Thus, the present invention relates to the modified crRNA comprising a 2′,3′-cyclic phosphate, and the methods for genome engineering based on the CRISPR/cas system (Jinek, Chylinski et al. 2012; Cong, Ran et al. 2013; Mali, Yang et al. 2013) comprising using the modified crRNA.

In a particular embodiment, the crRNA can be engineered to recognize at least the two target nucleic acid sequences simultaneously. In this case, same crRNA comprises at least two sequences complementary to a portion of the two target nucleic acid sequences. In a preferred embodiment, said complementary sequences are spaced by a repeat sequence.

Trans-activating CRISPR RNA according to the present invention are characterized by an anti-repeat sequence capable of base-pairing with at least a part of the 3′ extension sequence of crRNA to form a tracrRNA:crRNA also named guide RNA (gRNA). TracrRNA comprises a sequence complementary to a region of the crRNA.

A synthetic single guide RNA (sgRNA) comprising a fusion of crRNA and tracrRNA that forms a hairpin that mimics the tracrRNA-crRNA complex (Cong, Ran et al. 2013; Mali, Yang et al. 2013) can be used to direct Cas9 endonuclease-mediated cleavage of target nucleic acid. This system has been shown to function in a variety of eukaryotic cells, including human, zebra fish and yeast. The sgRNA may comprise two distinct sequences complementary to a portion of the two target nucleic acid sequences, preferably spaced by a repeat sequence.

The methods of the invention involve introducing crRNA, tracrRNA, sgRNA and Cas9 into a cell. crRNA, tracrRNA, sgRNA or Cas9 may be synthesized in situ in the cell as a result of the introduction of polynucleotide encoding RNA or polypeptides into the cell. Alternatively, the crRNA, tracRNA, sgRNA, Cas9 RNA or Cas9 polypeptides could be produced outside the cell and then introduced thereto. Methods for introducing a polynucleotide construct into bacteria, plants, fungi and animals are known in the art and including as non limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposomes and the like. For example, transient transformation methods include for example microinjection, electroporation or particle bombardment. Said polynucleotides may be included in vectors, more particularly plasmids or virus, in view of being expressed in prokaryotic or eukaryotic cells.

The invention also concerns the polynucleotides, in particular DNA or RNA encoding the polypeptides and proteins previously described. These polynucleotides may be included in vectors, more particularly plasmids or virus, in view of being expressed in prokaryotic or eukaryotic cells.

The present invention contemplates modification of the Cas9 polynucleotide sequence such that the codon usage is optimized for the organism in which it is being introduced. Thus, for example Cas9 polynucleotide sequence derived from the pyogenes or S. Thermophilus codon optimized for use in human is set forth in (Cong, Ran et al. 2013; Mali, Yang et al. 2013).

In particular embodiments, the Cas9 polynucleotides according to the present invention can comprise at least one subcellular localization motif. A subcellular localization motif refers to a sequence that facilitates transporting or confining a protein to a defined subcellular location that includes at least one of the nucleus, cytoplasm, plasma membrane, endoplasmic reticulum, golgi apparatus, endosomes, peroxisomes and mitochondria. Subcellular localization motifs are well-known in the art. A subcellular localization motif requires a specific orientation, e.g., N- and/or C-terminal to the protein. As a non-limiting example, the nuclear localization signal (NLS) of the simian virus 40 large T-antigen can be oriented at the N and/or C-terminus. NLS is an amino acid sequence which acts to target the protein to the cell nucleus through Nuclear Pore Complex and to direct a newly synthesized protein into the nucleus via its recognition by cytosolic nuclear transport receptors. Typically, a NLS consists of one or more short sequences of positively charged amino acids such as lysines or arginines.

The present invention also relates to a method for modifying genetic sequence of interest further comprising the step of expressing an additional catalytic domain into a host cell. In a more preferred embodiment, the present invention relates to a method to increase mutagenesis wherein said additional catalytic domain is a DNA end-processing enzyme. Non limiting examples of DNA end-processing enzymes include 5-3′ exonucleases, 3-5′ exonucleases, 5-3′ alkaline exonucleases, 5′ flap endonucleases, helicases, hosphatase, hydrolases and template-independent DNA polymerases. Non limiting examples of such catalytic domain comprise of a protein domain or catalytically active derivate of the protein domain selected from the group consisting of hExoI (EXO1_HUMAN), Yeast ExoI (EXO1_YEAST), E. coli ExoI, Human TREX2, Mouse TREX1, Human TREX1, Bovine TREX1, Rat TREX1, TdT (terminal deoxynucleotidyl transferase) Human DNA2, Yeast DNA2 (DNA2_YEAST). In a preferred embodiment, said additional catalytic domain has a 3′-5′-exonuclease activity, and in a more preferred embodiment, said additional catalytic domain has TREX exonuclease activity, more preferably TREX2 activity. In another preferred embodiment, said catalytic domain is encoded by a single chain TREX polypeptide.

Endonucleolytic breaks are known to stimulate the rate of homologous recombination. Therefore, in another preferred embodiment, the present invention relates to a method for inducing homologous gene targeting in the genetic sequence of interest further comprising providing to the cell an exogeneous nucleic acid comprising at least a sequence homologous to a portion of the genetic sequence of interest, such that homologous recombination occurs between the genetic sequence of interest and the exogenous nucleic acid.

In particular embodiments, said exogenous nucleic acid comprises first and second portions which are homologous to region 5′ and 3′ of the genetic sequence of interest respectively. Said exogenous nucleic acid in these embodiments also comprises a third portion positioned between the first and the second portion which comprises no homology with the regions 5′ and 3′ of the genetic sequence of interest. Following cleavage of the genetic sequence of interest, a homologous recombination event is stimulated between the target nucleic acid sequence and the exogenous nucleic acid. Preferably, homologous sequences of at least 50 bp, preferably more than 100 bp and more preferably more than 200 bp are used within said exogenous nucleic acid. Therefore, the exogenous nucleic acid is preferably from 200 bp to 6000 bp, more preferably from 1000 bp to 2000 bp. Indeed, shared nucleic acid homologies are located in regions flanking upstream and downstream the cleavage induced and the nucleic acid sequence to be introduced should be located between the two arms.

Depending on the location of the genetic sequence of interest wherein break event has occurred, such exogenous nucleic acid can be used to knock-out a gene, e.g. when exogenous nucleic acid is located within the open reading frame of said gene, or to introduce new sequences or genes of interest. Sequence insertions by using such exogenous nucleic acid can be used to modify a targeted existing gene, by correction or replacement of said gene (allele swap as a non-limiting example), or to up- or down-regulate the expression of the targeted gene (promoter swap as non-limiting example), said targeted gene correction or replacement.

### Modified Cells and Kits

A variety of cells are suitable for use in the method according to the invention. Cells can be any prokaryotic or eukaryotic living cells, cell lines derived from these organisms for in vitro cultures, primary cells from animal or plant origin.

By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines. These cells thus represent a more valuable model to the in vivo state they refer to.

In the frame of the present invention, “eukaryotic cells” refer to a fungal, plant, algal or animal cell or a cell line derived from the organisms listed below and established for in vitro culture. More preferably, the fungus is of the genus Aspergillus, Penicillium, Acremonium, Trichoderma, Chrysoporium, Mortierella, Kluyveromyces or Pichia; More preferably, the fungus is of the species Aspergillus niger, Aspergillus nidulans, Aspergillus oryzae, Aspergillus terreus, Penicillium chrysogenum, Penicillium citrinum, Acremonium Chrysogenum, Trichoderma reesei, Mortierella alpine, Chrysosporium lucknowense, Kluyveromyceslactis, Pichia pastoris or Pichia ciferrii. More preferably the plant is of the genus Arabidospis, Nicotiana, Solanum, lactuca, Brassica, Oryza, Asparagus, Pisum, Medicago, Zea, Hordeum, Secale, Triticum, Capsicum, Cucumis, Cucurbita, Citrullis, Citrus, Sorghum; More preferably, the plant is of the species Arabidospis thaliana, Nicotiana tabaccum, Solanum lycopersicum, Solanum tuberosum, Solanum melongena, Solanum esculentum, Lactuca saliva, Brassica napus, Brassica oleracea, Brassica rapa, Oryza glaberrima, Oryza sativa, Asparagus officinalis, Pisumsativum, Medicago sativa, zea mays, Hordeum vulgare, Secale cereal, Triticuma estivum, Triticum durum, Capsicum sativus, Cucurbitapepo, Citrullus lanatus, Cucumis melo, Citrus aurantifolia, Citrus maxima, Citrus medico, Citrus reticulata. More preferably the animal cell is of the genus Homo, Rattus, Mus, Sus, Bos, Danio, Canis, Felis, Equus, Salmo, Oncorhynchus, Gallus, Meleagris, Drosophila, Caenorhabditis; more preferably, the animal cell is of the species Homo sapiens, Rattus norvegicus, Mus musculus, Sus scrofa, Bos taurus, Danio rerio, Canis lupus, Felis catus, Equus caballus, Salmo solar, Oncorhynchus mykiss, Gallus gallus, Meleagris gallopavo, Drosophila melanogaster, Caenorhabditis elegans.

In the present invention, the cell is preferably a plant cell, a mammalian cell, a fish cell, an insect cell or cell lines derived from these organisms for in vitro cultures or primary cells taken directly from living tissue and established for in vitro culture. As non limiting examples cell lines can be selected from the group consisting of CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells. Are also encompassed in the scope of the present invention stem cells, embryonic stem cells and induced Pluripotent Stem cells (iPS).

All these cell lines can be modified by the method of the present invention to provide cell line models to produce, express, quantify, detect, study a gene or a protein of interest; these models can also be used to screen biologically active molecules of interest in research and production and various fields such as chemical, biofuels, therapeutics and agronomy as non-limiting examples. A particular aspect of the present invention relates to an isolated cell as previously described obtained by the method according to the invention. Typically, said isolated cell comprises Cas9 nickases, crRNA(s) and tracrRNA or sgRNA. Resulting isolated cell comprises a modified genetic sequence of interest in which a cleavage has occurred. The resulting modified cell can be used as a cell line for a diversity of applications ranging from bioproduction, animal transgenesis (by using for instance stem cells), plant transgenesis (by using for instance protoplasts), to cell therapy (by using for instance T-cells). The methods of the invention are useful to engineer genomes and to reprogram cells, especially iPS cells and ES cells. Another aspect of the invention is a kit for cell transformation comprising one or several of the components of the modified type II CRISPR system according to the invention as previously described. This kit more particularly comprises:
two crRNAs comprising a sequence complementary to one strand of a first and second double-strand nucleic acid target sequences comprising PAM motif in the other strand and having a 3′ extension sequence;
at least one tracrRNA comprising a sequence complementary to the 3′ extension sequences of said crRNAs;
at least one cas9 nuclease harboring a non-functional RuvC-like or HNH nuclease domain or a polynucleotide encoding thereof.

In another embodiment, the kit comprises:
Two crRNAs comprising a sequence complementary to one strand of a first and second double-strand nucleic acid target sequences comprising different PAM motifs in the other strand and having a 3′ extension sequence;
at least one tracrRNA comprising a sequence complementary to the 3′ extension sequences of said crRNAs;
a first Cas9 nuclease specifically recognizing the PAM motif of the first nucleic acid target and harboring a non-functional RuvC-like or a polynucleotide encoding thereof.
a second Cas9 nuclease specifically recognizing the PAM motif of the second nucleic acid target and harboring a non-functional HNH nuclease domain or a polynucleotide encoding thereof.

### Method for Generating an Animal/a Plant

The present invention also encompasses transgenic animals or plants which comprises modified targeted genetic sequence of interest by the methods described above. Animals may be generated by methods described above into a cell or an embryo. In particular, the present invention relates to a method for generating an animal, comprising providing an eukaryotic cell comprising a genetic sequence of interest into which it is desired to introduce a genetic modification; generating a cleavage within the genetic sequence of interest by any one of the methods according to the present invention; and generating an animal from the cell or progeny thereof, in which cleavage has occurred. Typically, the embryo is a fertilized one cell stage embryo. Components of the method may be introduced into the cell by any of the methods known in the art including micro injection into the nucleus or cytoplasm of the embryo. In a particular embodiment, the method for generating an animal, further comprise introducing an exogenous nucleic acid as desired. The exogenous nucleic acid can include for example a nucleic acid sequence that disrupts a gene after homologous recombination, a nucleic acid sequence that replaces a gene after homologous recombination, a nucleic acid sequence that introduces a mutation into a gene after homologous recombination or a nucleic acid sequence that introduce a regulatory site after homologous recombination. The embryos are then cultures to develop an animal. In one aspect of the invention, an animal in which at least a genetic sequence of interest has been engineered is provided. For example, an engineered gene may become inactivated such that it is not transcribed or properly translated, or an alternate form of the gene is expressed. The animal may be homozygous or heterozygous for the engineered gene.

The present invention also related to a method for generating a plant comprising providing a plant cell comprising a genetic sequence of interest into which it is desired to introduce a genetic modification; generating a cleavage within the genetic sequence of interest by any one of the methods according to the present invention; and generating a plant from the cell or progeny thereof, in which cleavage has occurred. Progeny includes descendants of a particular plant or plant line. In a particular embodiment, the method for generating a plant, further comprise introducing an exogenous nucleic acid as desired. Plant cells produced using methods can be grown to generate plants having in their genome a modified genetic locus of interest. Seeds from such plants can be used to generate plants having a phenotype such as, for example, an altered growth characteristic, altered appearance, or altered compositions with respect to unmodified plants.

### Therapeutic Applications

The method disclosed herein can have a variety of applications. In one embodiment, the method can be used for clinical or therapeutic applications. The method can be used to repair or correct disease-causing genes, as for example a single nucleotide change in sickle-cell disease. The method can be used to correct splice junction mutations, deletions, insertions, and the like in other genes or chromosomal sequences that play a role in a particular disease or disease state.

Such methods can also be used to genetically modify iPS or primary cells, for instance T-cells, in view of injected such cells into a patient for treating a disease or infection. Such cell therapy schemes are more particularly developed for treating cancer, viral infection such as caused by CMV or HIV or self-immune diseases.

### DEFINITIONS

In the description above, a number of terms are used extensively. The following definitions are provided to facilitate understanding of the present embodiments.

As used herein, “a” or “an” may mean one or more than one.

Amino acid residues in a polypeptide sequence are designated herein according to the one-letter code, in which, for example, Q means Gln or Glutamine residue, R means Arg or Arginine residue and D means Asp or Aspartic acid residue.

Amino acid substitution means the replacement of one amino acid residue with another, for instance the replacement of an Arginine residue with a Glutamine residue in a peptide sequence is an amino acid substitution.

Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.

As used herein, “nucleic acid” or polynucleotide” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

By “complementary sequence” is meant the sequence part of polynucleotide (e.g. part of crRNa or tracRNA) that can hybridize to another part of polynucleotides (e.g. the target nucleic acid sequence or the crRNA respectively) under standard low stringent conditions. Such conditions can be for instance at room temperature for 2 hours by using a buffer containing 25% formamide, 4×SSC, 50 mM NaH2PO4/Na2HPO4 buffer; pH 7.0, 5×Denhardt's, 1 mM EDTA, 1 mg/ml DNA+20 to 200 ng/ml probe to be tested (approx. 20-200 ng/ml)). This can be also predicted by standard calculation of hybridization using the number of complementary bases within the sequence and the content in G-C at room temperature as provided in the literature. Preferentially, the sequences are complementary to each other pursuant to the complementarity between two nucleic acid strands relying on Watson-Crick base pairing between the strands, i.e. the inherent base pairing between adenine and thymine (A-T) nucleotides and guanine and cytosine (G-C) nucleotides. Accurate base pairing equates with Watson-Crick base pairing includes base pairing between standard and modified nucleosides and base pairing between modified nucleosides, where the modified nucleosides are capable of substituting for the appropriate standard nucleosides according to the Watson-Crick pairing. The complementary sequence of the single-strand oligonucleotide can be any length that supports specific and stable hybridization between the two single-strand oligonucleotides under the reaction conditions. The complementary sequence generally authorizes a partial double stranded overlap between the two hybridized oligonucleotides over more than 3 bp, preferably more than 5 bp, preferably more than to 10 bp. The complementary sequence is advantageously selected not to be homologous to any sequence in the genome to avoid off-target recombination or recombination not involving the whole exogenous nucleic acid sequence (i.e. only one oligonucleotide).

By “nucleic acid homologous sequence” it is meant a nucleic acid sequence with enough identity to another one to lead to homologous recombination between sequences, more particularly having at least 80% identity, preferably at least 90% identity and more preferably at least 95%, and even more preferably 98% identity. “Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting.

The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e. g. adenoassociated viruses), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Having generally described this invention, a further understanding can be obtained by reference to certain specific examples, which are provided herein for purposes of illustration only, and are not intended to be limiting unless otherwise specified.

### REFERENCES

Chylinski, K., A. Le Rhun, et al. (2013). “The tracrRNA and Cas9 families of type II CRISPR-Cas immunity systems.” RNA Biol 10(5).
Cong, L., F. A. Ran, et al. (2013). “Multiplex genome engineering using CRISPR/Cas systems.” Science 339(6121): 819-23.
Critchlow, S. E. and S. P. Jackson (1998). “DNA end-joining: from yeast to man.” Trends Biochem Sci 23(10): 394-8.
Dalgaard, J. Z., A. J. Klar, et al. (1997). “Statistical modeling and analysis of the LAGLIDADG family of site-specific endonucleases and identification of an intein that encodes a site-specific endonuclease of the HNH family.” Nucleic Acids Res 25(22): 4626-38.
Deltcheva, E., K. Chylinski, et al. (2011). “CRISPR RNA maturation by trans-encoded small RNA and host factor RNase III.” Nature 471(7340): 602-7.
Deveau, H., R. Barrangou, et al. (2008). “Phage response to CRISPR-encoded resistance in Streptococcus thermophilus.” J Bacteriol 190(4): 1390-400.
Garneau, J. E., M. E. Dupuis, et al. (2010). “The CRISPR/Cas bacterial immune system cleaves bacteriophage and plasmid DNA.” Nature 468(7320): 67-71.
Gasiunas, G., R. Barrangou, et al. (2012). “Cas9-crRNA ribonucleoprotein complex mediates specific DNA cleavage for adaptive immunity in bacteria.” Proc Natl Acad Sci USA 109(39): E2579-86.
Gorbalenya, A. E. (1994). “Self-splicing group I and group II introns encode homologous (putative) DNA endonucleases of a new family.” Protein Sci 3(7): 1117-20.
Haft, D. H., J. Selengut, et al. (2005). “A guild of 45 CRISPR-associated (Cas) protein families and multiple CRISPR/Cas subtypes exist in prokaryotic genomes.” PLoS Comput Biol 1(6): e60.
Horvath, P. and R. Barrangou (2010). “CRISPR/Cas, the immune system of bacteria and archaea.” Science 327(5962): 167-70.
Jiang, W., D. Bikard, et al. (2013). “RNA-guided editing of bacterial genomes using CRISPR-Cas systems.” Nat Biotechnol 31(3): 233-9.
Jinek, M., K. Chylinski, et al. (2012). “A programmable dual-RNA-guided DNA endonuclease in adaptive bacterial immunity.” Science 337(6096): 816-21.
Kleanthous, C., U. C. Kuhlmann, et al. (1999). “Structural and mechanistic basis of immunity toward endonuclease colicins.” Nat Struct Biol 6(3): 243-52.
Ma, J. L., E. M. Kim, et al. (2003). “Yeast Mre11 and Rad1 proteins define a Ku-independent mechanism to repair double-strand breaks lacking overlapping end sequences.” Mol Cell Biol 23(23): 8820-8.
Makarova, K. S., N. V. Grishin, et al. (2006). “A putative RNA-interference-based immune system in prokaryotes: computational analysis of the predicted enzymatic machinery, functional analogies with eukaryotic RNAi, and hypothetical mechanisms of action.” Biol Direct 1: 7.
Mali, P., L. Yang, et al. (2013). “RNA-guided human genome engineering via Cas9.” Science339(6121): 823-6.
Mojica, F. J., C. Diez-Villasenor, et al. (2009). “Short motif sequences determine the targets of the prokaryotic CRISPR defence system.” Microbiology 155(Pt 3): 733-40.
Qi, L. S., M. H. Larson, et al. (2013). “Repurposing CRISPR as an RNA-guided platform for sequence-specific control of gene expression.” Cell 152(5): 1173-83.
Sapranauskas, R., G. Gasiunas, et al. (2011). “The Streptococcus thermophilus CRISPR/Cas system provides immunity in Escherichia coli.” Nucleic Acids Res 39(21): 9275-82.
Schutz, K., J. R. Hesselberth, et al. (2010). “Capture and sequence analysis of RNAs with terminal 2′,3′-cyclic phosphates.” Rna 16(3): 621-31.
Shah, S. A., S. Erdmann, et al. (2013). “Protospacer recognition motifs: Mixed identities and functional diversity.” RNA Biol 10(5).
Shub, D. A., H. Goodrich-Blair, et al. (1994). “Amino acid sequence motif of group I intron endonucleases is conserved in open reading frames of group II introns.” Trends Biochem Sci 19(10): 402-4.
Sorek, R., C. M. Lawrence, et al. (2013). “CRISPR-mediated Adaptive Immune Systems in Bacteria and Archaea.” Annu Rev Biochem.
van der Ploeg, J. R. (2009). “Analysis of CRISPR in Streptococcus mutans suggests frequent occurrence of acquired immunity against infection by M102-like bacteriophages.” Microbiology155(Pt 6): 1966-76.

1-20. (canceled)

21. A method for precisely inducing a nucleic acid cleavage in a genetic sequence in a cell comprising:
(a) Selecting a first and second double-stranded nucleic acid targets in said genetic sequence, each nucleic acid targets comprising, on one strand, a protospacer adjacent motif (PAM) at one 3′ extremities;
(b) engineering two CRISPR targeting RNA (crRNAs) comprising each:
a sequence complementary to one part of the opposite strand of the nucleic acid target that does not comprise the PAM motif, and
a 3′ extension sequence;
(c) providing at least one trans-activating CRISPR targeting RNA (tracrRNA) comprising a sequence complementary to one part of the 3′ extension sequences of said crRNAs under b);
(d) providing at least one cas9 nickase harboring either a non-functional RuvC-like or a non-functional HNH nuclease domain and recognizing said PAM motif(s);
(e) introducing into the cell said crRNAs, said tracrRNA(s) and said Cas9 nickase;
such that each Cas9-tracrRNA:crRNA complex induces a nick event in double-stranded nucleic acid targets in order to cleave the genetic sequence between said first and second nucleic acid targets.

22. The method of claim 21, wherein the two PAM motifs are present on opposed nucleic acid strands.

23. The method of claim 21, wherein the two PAM motifs are present on the same nucleic acid strand.

24. The method according to claim 21 wherein the first and second double-stranded nucleic acid targets comprise different PAM motifs specifically recognized by two different Cas9 nickases.

25. The method of claim 24, wherein said method involves a first Cas9 nickase harboring a non-functional RuvC-like and a second Cas9 nickase harboring a non-functional HNH nuclease domain.

26. The method according to claim 21, wherein at least one Cas9 nickase comprises at least one mutation in the RuvC domain.

27. The method according to claim 21, wherein at least one Cas 9 nickase comprises at least one mutation in the HNH domain.

28. The method according to claim 21, wherein each crRNA comprises complementary sequence from 12 to 20 nucleotides.

29. The method according to claim 21, comprising in step b) engineering one crRNA comprising two sequences complementary to a part of each target nucleic acid sequences.

30. The method according to claim 21, wherein the crRNA and the tracrRNA are fused to form a single guide RNA.

31. The method according to claim 21, wherein the first and the second nucleic acid target sequences are spaced from each other by a spacer region from 1 to 300 bp, preferably from 3 to 250 bp.

32. The method according to claim 21, further comprising introducing an exogenous nucleic acid sequence comprising at least one sequence homologous to at least a portion of the genetic sequence, such that homologous recombination occurs between said exogenous sequence and genetic sequence.

33. The method of claim 21, wherein the cell is a plant cell.

34. The method of claim 21, wherein the cell is a mammalian cell.

35. The method according to claim 34, wherein said cell is a primary T-cell.

36. An isolated cell comprising:
two crRNAs comprising sequences complementary to a first and second double-strand nucleic acid target sequences and having a 3′ extension sequence;
at least one tracrRNA comprising a sequence complementary to the 3′ extension sequences of said crRNAs;
at least one cas9 nickase or a polynucleotide encoding thereof.

37. A kit for precisely inducing a nucleic acid cleavage in a genetic sequence in a cell comprising:
two crRNAs comprising a sequence complementary to a first and second double-strand nucleic acid target sequences having a 3′ extension sequence;
at least one tracrRNA comprising a sequence complementary to the 3′ extension sequences of said crRNAs;
at least one cas9 nickase or a polynucleotide encoding thereof.

38. A method for generating an animal comprising:
(a) providing a eukaryotic cell comprising a genetic sequence into which it is desired to introduce a genetic modification;
(b) inducing cleavage within said genetic sequence by the method according to claim 21; and
(c) generating an animal from the cell or progeny thereof, in which a nucleic acid cleavage has occurred.

39. A method of claim 38, further comprising: introducing into the cell an exogenous nucleic acid comprising a sequence homologous to at least a portion of the target nucleic acid sequence and generating an animal from the cell or progeny thereof in which homologous recombination has occurred.

40. A method for generating a plant comprising:
(d) providing a plant cell comprising a genetic sequence into which it is desired to introduce a genetic modification;
(e) inducing nucleic acid cleavage within said genetic sequence cell by the method according to claim 21; and
(f) generating a plant from the cell or progeny thereof in which a nucleic acid cleavage has occurred.

41. The method of claim 40 further comprising: introducing into the plant cell an exogenous nucleic acid comprising a sequence homologous to at least a portion of the target nucleic acid sequence; and generating a plant from the cell or progeny thereof in which homologous recombination has occurred.


SEQUENCE LISTING (1 sequence)

SEQ ID NO: 1 - protein, 1368 aa, Streptococcus pyogenes serotype M1 [sequence omitted]